SEQUENCE LISTING (2 sequences)

SEQ ID NO: 1 - protein, 238 aa, synthetic construct
  REGION 1..238: note = chimeric L chain
MESQTHVLISLLLSVSGTYGDIAITQSPSSVAVSVGETVTLSCKSSQSLLYSENQKDYLGWYQQKPGQTPKPLIYWATNRHTGVPDRFTGSGSGTDFTLIISSVQAEDLADYYCGQYLVYPFTFGPGTKLELKQPKASPSVTLFPPSSEELGANKATLVCLISDFYPSGVTVAWKASGSPVTQGVETTKPSKQSNNKYAASSYLSLTPDKWKSHSSFSCLVTHEGSTVEKKVAPAECS

SEQ ID NO: 2 - protein, 468 aa, synthetic construct
  REGION 1..468: note = chimeric H chain
MGWSQIILFLVAAATCVHSQVQLQQSGAELVKPGSSVKISCKASGYTFTSNFMHWVKQQPGNGLEWIGWIYPEYGNTKYNQKFDGKATLTADKSSSTAYMQLSSLTSEDSAVYFCASEEAVISLVYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

(19) United States
(12) Patent Application Publication
(10) Pub. No.: US 2026/0258134 A1
(43) Pub. Date: Sep. 3, 2026

(54) ENHANCING ANTI-TUMOR IMMUNITY

(71) Applicant: NATIONAL UNIVERSITY CORPORATION HOKKAIDO UNIVERSITY, Sapporo-shi, Hokkaido (JP)

(72) Inventors: Satoru KONNAI, Sapporo-shi, Hokkaido (JP); Naoya MAEKAWA, Sapporo-shi, Hokkaido (JP); Tomohiro OKAGAWA, Sapporo-shi, Hokkaido (JP); Kazuhiko OHASHI, Sapporo-shi, Hokkaido (JP); Shiro MURATA, Sapporo-shi, Hokkaido (JP); Yasuhiko SUZUKI, Sapporo-shi, Hokkaido (JP)

(73) Assignee: NATIONAL UNIVERSITY CORPORATION HOKKAIDO UNIVERSITY, Sapporo-shi, Hokkaido (JP)

(21) Appl. No.: 18/876,188

(22) PCT Filed: May 31, 2023
(86) PCT No.: PCT/JP2023/020232

§ 371 Date: Dec. 17, 2024

(30) Foreign Application Priority Data
Jun. 20, 2022 (JP) 2022-099097

(51) Int. Cl. C07K 16/28; A61K 39/00; A61N 5/10; A61P 35/00

(52) U.S. Cl. CPC C07K 16/2827; A61P 35/00; A61K 2039/505; A61N 5/10; C07K 2317/24

(57) ABSTRACT

Provided is a therapy which is capable of achieving a better response rate and/or survival benefit in non-human mammals with cancer.

A method of treating cancer in non-human mammals, comprising initiating treatment with a PD-1/PD-L1 inhibitor after conducting radiation therapy. A pharmaceutical composition for treating cancer in non-human mammals, wherein the composition comprises a PD-1/PD-L1 inhibitor and administration of the composition is initiated after conducting radiation therapy. A PD-1/PD-L1 inhibitor for use in a method of treating cancer in non-human mammals, comprising initiating treatment with the PD-1/PD-L1 inhibitor after conducting radiation therapy.

### TECHNICAL FIELD

The present invention relates to enhancement of anti-tumor immunity.

### BACKGROUND ART

The number of dogs dying of malignant tumor (cancer) has been increasing recently due to the increase of lifespan in dogs, and development of new therapeutic strategies in addition to existing therapies is desired. In human medicine, application of immunotherapy in addition to surgery, radiation and chemotherapy has progressed. In particular, immune checkpoint inhibitors (ICIs), such as anti-programmed cell death 1 (PD-1) antibody and anti-PD-ligand 1 (PD-L1) antibody, have been reported to produce good therapeutic results against many cancer species including malignant melanoma and lung cancer (Postow et al., 2015 (Non-Patent Document No. 1); Ribas, 2015 (Non-Patent Document No. 2)). To date, clinical studies conducted by the present inventors at the Hokkaido University Veterinary Teaching Hospital have revealed that immune suppression via PD-1/PD-L1 pathway also occurs in canine malignant tumor (Maekawa et al., 2014 and 2016 (Non-Patent Documents Nos. 3 and 4)), and that a canine chimeric anti-PD-L1 antibody, the world's first immune checkpoint inhibitor for dogs, induces tumor regression in some dogs (Maekawa et al., 2017 and 2021 (Non-Patent Documents Nos. 5 and 6)). However, further improvement is required in the response rate of the canine chimeric anti-PD-L1 antibody.

### PRIOR ART LITERATURE

### Non-Patent Documents

Non-Patent Document No. 1: Postow M A, Callahan M K, Wolchok J D., Immune Checkpoint Blockade in Cancer Therapy. J Clin Oncol. 2015 Jun. 10; 33(17): 1974-82. doi: 10.1200/JCO.2014.59.4358
Non-Patent Document No. 2: Ribas A., Releasing the Brakes on Cancer Immunotherapy. N Engl J Med. 2015 Oct. 15; 373 (16): 1490-2. doi: 10.1056/NEJMp1510079.
Non-Patent Document No. 3: Maekawa N, Konnai S, Ikebuchi R, Okagawa T, Adachi M, Takagi S, Kagawa Y, Nakajima C, Suzuki Y, Murata S, Ohashi K., Expression of PD-L1 on canine tumor cells and enhancement of IFN-γ production from tumor-infiltrating cells by PD-L1 blockade. PLOS One. 2014 Jun. 10; 9 (6): e98415. doi: 10.1371/journal.pone.0098415.
Non-Patent Document No. 4: Maekawa N, Konnai S, Okagawa T, Nishimori A, Ikebuchi R, Izumi Y, Takagi S, Kagawa Y, Nakajima C, Suzuki Y, Kato Y, Murata S, Ohashi K., Immunohistochemical Analysis of PD-L1 Expression in Canine Malignant Cancers and PD-1 Expression on Lymphocytes in Canine Oral Melanoma. PLOS One. 2016 Jun. 8; 11 (6): e0157176. doi: 10.1371/journal.pone.0157176.
Non-Patent Document No. 5: Maekawa N, Konnai S, Takagi S, Kagawa Y, Okagawa T, Nishimori A, Ikebuchi R, Izumi Y, Deguchi T, Nakajima C, Kato Y, Yamamoto K, Uemura H, Suzuki Y, Murata S, Ohashi K., A canine chimeric monoclonal antibody targeting PD-L1 and its clinical efficacy in canine oral malignant melanoma or undifferentiated sarcoma. Sci Rep. 2017 Aug. 21; 7 (1): 8951. doi: 10.1038/s41598-017-09444-2.
Non-Patent Document No. 6: Maekawa N, Konnai S, Nishimura M, Kagawa Y, Takagi S, Hosoya K, Ohta H, Kim S, Okagawa T, Izumi Y, Deguchi T, Kato Y, Yamamoto S, Yamamoto K, Toda M, Nakajima C, Suzuki Y, Murata S, Ohashi K., PD-L1 immunohistochemistry for canine cancers and clinical benefit of anti-PD-L1 antibody in dogs with pulmonary metastatic oral malignant melanoma. NPJ Precis Oncol. 2021 Feb. 12; 5 (1): 10. doi: 10.1038/s41698-021-00147-6.

### DISCLOSURE OF THE INVENTION

### Problem for Solution by the Invention

Hypofractionated radiation therapy is an effective local treatment for canine malignant melanoma. Recent studies in humans have suggested that radiation therapy (RT) combined with ICIs induces robust systemic antitumor immunity in patients with cancer since RT initiates antitumor immune responses via induction of immunogenic cell death (ICD) in tumor tissues.

It is an object of the present invention to provide a therapy capable of achieving a better response rate and/or survival benefit in non-human mammals with cancer.

### Means to Solve the Problem

To investigate whether combination of RT and anti-PD-L1 antibody induces better systemic antitumor immunity in dogs with malignant melanoma, the present inventors retrospectively evaluated the response rate of intrathoracic metastatic lesions and overall survival (OS) in dogs with pulmonary metastatic oral malignant melanoma (OMM) (n=39) treated with a canine chimeric anti-PD-L1 antibody (c4G12). In “No RT group” (n=20) consisting of dogs which were considered free from the effect of RT, intrathoracic clinical benefit rate (CBR) was 10.0% (2/20) and median OS was 185 days. In “Previous RT group” (n=9) consisting of dogs which received RT within 8 weeks prior to the first dose of c4G12, CBR was 55.6% (5/9, P=0.016 vs. No RT group) and median OS was 283.5 days (P=0.036 vs. No RT group). In “Concurrent RT group” (n=10) in which c4G12 treatment started within ±1 week of the first fraction of RT, CBR was 20.0% (2/10) and median OS was 129 days, with no significant difference compared to No RT group. In the combination therapy, adverse events equivalent to those observed in each monotherapy were observed. Taken together, hypofractionated RT before the initiation of c4G12 treatment can be an effective approach to enhance the therapeutic efficacy of immunotherapy with acceptable safety profiles. The present invention has been achieved based on these findings.

The gist of the present invention is as follows.
(1) A method of treating cancer in non-human mammals, comprising initiating treatment with a PD-1/PD-L1 inhibitor after conducting radiation therapy.
(2) A pharmaceutical composition for treating cancer in non-human mammals, wherein the composition comprises a PD-1/PD-L1 inhibitor and administration of the composition is initiated after conducting radiation therapy.
(3) A PD-1/PD-L1 inhibitor for use in a method of treating cancer in non-human mammals, comprising initiating treatment with the PD-1/PD-L1 inhibitor after conducting radiation therapy.

### Effect of the Invention

According to the present invention, it is possible to enhance the efficacy of cancer immunotherapy for non-human mammals with cancer.

The present specification encompasses the contents of the specification and/or drawings disclosed in Japanese Patent Application No. 2022-99097 based on which the present application claims priority.

### BRIEF DESCRIPTION OF THE DRAWINGS

FIG. 1 Grouping of dogs based on the absence or presence of RT, as well as the timing of RT, and the overall survival in c4G12 immunotherapy. (A) The timing of RT and c4G12 immunotherapy in each treatment group. (B) Kaplan-Meier curves showing OS from the initiation of c4G12 immunotherapy. Marks on the line indicate censored data. Statistical analysis was performed using the log-rank test.

FIG. 2 The overall survival of dogs with pulmonary metastatic OMM treated by RT only or by c4G12 immunotherapy and RT concurrently. Kaplan-Meier curves showing OS from the initiation of RT. Marks on the line indicate censored data. Statistical analysis was performed using the log-rank test.

### BEST MODES FOR CARRYING OUT THE INVENTION

Hereinafter, embodiments of the present invention will be described in more detail.

The present invention provides a method of treating cancer in non-human mammals, comprising initiating treatment with a PD-1/PD-L1 inhibitor after conducting radiation therapy.

The present invention also provides a pharmaceutical composition for treating cancer in non-human mammals, wherein the composition comprises a PD-1/PD-L1 inhibitor and administration of the composition commences after conducting radiation therapy.

Further, the present invention provides a PD-1/PD-L1 inhibitor for use in a method of treating cancer in non-human mammals, comprising initiating treatment with the PD-1/PD-L1 inhibitor after conducting radiation therapy.

In the present invention, the target disease to be treated is cancer. Specific examples of cancer include, but are not limited to, malignant melanoma, pulmonary cancer, stomach cancer, kidney cancer, breast cancer (mammary tumor), bladder cancer, esophageal cancer, ovarian cancer, prostate cancer, squamous cell carcinoma, anal sac gland carcinoma, intranasal adenocarcinoma, soft tissue sarcoma, osteosarcoma, angiosarcoma (hemangiosarcoma), histiocytic sarcoma, mastocytoma (mast cell tumor), lymphoma and leukemia. Cancer may have distant metastasis, such as lymph node metastasis or lung metastasis. Sites where malignant melanoma may develop include, but are not limited to, oral cavity, nasal cavity, hair bearing skin, mucocutaneous junction, nail bed, foot pad, eyeball, digestive tract and anal sac.

In the present invention, target non-human mammals to be treated include, but are not limited to, dog, cat, sheep, goat, pig, horse, cattle, water buffalo, yak, rabbit, mouse, rat, hamster and guinea pig.

Radiation therapy is a therapy that kills cancer cells by exposing them to radiation. There are two types of radiation therapy: external irradiation, which applies radiation from outside the body, and internal irradiation, which applies radiation to cancer or its surrounding area from inside the body; both may be used in combination. Types of radiation include electromagnetic radiation (γ ray, X ray, etc.) and particle radiation (α ray, β ray, electron beam, proton beam, heavy ion beam, neutron beam, etc.). Cells, either normal tissues or cancer cells, exposed to radiation are damaged in the same manner. However, since normal tissues have a better recovery function than cancer cells, normal tissues recover over time, whereas cancer cells suffer from greater damage as damage to cancer cells accumulates over time. By conducting radiation therapy repeatedly (fractionated irradiation) taking advantage of the above-mentioned characteristic, normal tissues can be restored and cancer cells destroyed. Thus, the possibility of radical cure can be enhanced. Radiation therapy is an effective treatment for primary oral malignant melanoma and involved local lymph nodes. Hypofractionated irradiation protocols (e.g., 3-6 once-weekly fractions of 6-9 Gy) have often been used to treat canine oral malignant melanoma (OMM). In the Example described later, four fractions were performed at one week intervals (approximately 26-32 Gy in total). Cancers other than oral malignant melanoma may also be treated with the dose, frequency and the number of fractions based on these protocols.

In the present invention, treatment with a PD-1/PD-L1 inhibitor commences after conducting radiation therapy. As to PD-1/PD-L1 inhibitors, substances that specifically bind to PD-1 or PD-L1 may be given. Specific examples of such substances include, but are not limited to, proteins, polypeptides, oligopeptides, nucleic acids (including natural nucleic acids and artificial nucleic acids), low molecular weight organic compounds, inorganic compounds, cell extracts and extracts from plants/animals or soils. The substance may be either natural or synthetic. Preferable PD-1/PD-L1 inhibitors are antibodies, more preferably antibodies such as anti-PD-1 antibody, anti-PD-L1 antibody, etc. The antibody may be any antibody as long as it has inhibitory activity targeting PD-1/PD-L1, and may be any of the following types: polyclonal antibody, monoclonal antibody, chimeric antibody, single chain antibody, non-human mammalized antibody and non-human mammal-type antibody. Methods for preparing these antibodies are known. The antibody may be derived from any organism such as human, mouse, rat, rabbit, goat, guinea pig, dog or cattle. As used herein, the term “antibody” is a concept encompassing antibodies of smaller molecular sizes such as Fab, F(ab)′₂, ScFv, Diabody, V_{H}, V_{L}, Sc(F_{V})₂, Bispecific sc(F_{V})₂, Minibody, scFv-Fc monomer and scFv-Fc dimer. In the Example described later, a rat-dog chimeric anti-PD-L1 antibody designated c4G12 (Non-Patent Document No. 5) was used as a PD-1/PD-L1 inhibitor. c4G12 is a chimeric antibody in which a variable region gene of rat anti-bovine PD-L1 monoclonal antibody (4G12) capable of inhibiting the binding of canine PD-1 and PD-L1 is combined with a constant region gene of canine immunoglobulin (IgG-D). The amino acid sequence of the light chain (L chain) of c4G12 is shown in SEQ ID NO: 1, and the amino acid sequence of the heavy chain (H chain) of c4G12 in SEQ ID NO: 2. A method of preparing c4G12 is disclosed in Japanese Patent No. 6960634.

The timing of initiating the administration of PD-1/PD-L1 inhibitor may be after conducting RT. Preferably, the administration of PD-1/PD-L1 inhibitor is initiated within 0 day to 8 weeks after conducting RT. More preferably, the administration of PD-1/PD-L1 inhibitor is initiated within 1 to 8 weeks after conducting RT.

The pharmaceutical composition of the present invention is administered to subject animals systemically or locally and orally or parenterally.

The PD-1/PD-L1 inhibitor may be dissolved in buffer such as PBS, physiological saline, sterilized water or the like, sterilized through a filter if necessary, and then administered to subject animals by injection. Further, additives (such as coloring agents, emulsifiers, suspending agents, surfactants, solubilizers, stabilizers, preservatives, antioxidants, buffering agents, isotonic agents, pH adjusters, etc.) may be added to the solution of PD-1/PD-L1 inhibitor. As to administration routes, intravenous, intramuscular, intraperitoneal, subcutaneous or intradermal administration or the like may be enumerated. Transnasal or oral administration may also be used.

The content of the PD-1/PD-L1 inhibitor in a preparation varies with the type of the preparation and is usually 1-100% by weight, preferably 50-100% by weight. Such a preparation may be formulated into a unit dosage form.

The dose, number of administration and frequency of the PD-1/PD-L1 inhibitor (e.g., anti-PD-L1 antibody) may vary with the symptoms, age and body weight of the animal subject, the method of administration, dosage form and more. For example, usually, 0.1 to 100 mg/kg body weight, preferably 1-10 mg/kg body weight, may be administered per adult animal at least once at a frequency that enables confirmation of desired effect. For example, intravenous administration of c4G12 at a dose of 5 mg/kg (or 2 mg/kg) was performed 1 to 54 times at an interval of 2 weeks in the Example described later. The PD-1/PD-L1 inhibitor may be administered according to an administration schedule based on this dose, frequency and the number of administration.

### EXAMPLES

Hereinafter, the present invention will be described in detail based on the following Example.

### [Example 1] Enhanced Systemic Antitumor Immunity by Hypofractionated Radiotherapy and Anti-PD-L1 Therapy in Dogs with Pulmonary Metastatic Oral Malignant Melanoma

### Summary

Although checkpoint inhibitors (ICIs) have been developed to treat canine malignant melanoma and they achieved clinical results to some extent, desirable clinical efficacies have not been achieved yet. Hypofractionated radiation therapy is an effective local treatment for canine malignant melanoma. Recent studies in humans have suggested that radiation therapy (RT) combined with ICIs induces robust systemic antitumor immunity in patients with cancer since RT initiates antitumor immune responses via induction of immunogenic cell death (ICD) in tumor tissues. In the present invention, in order to investigate whether combination of RT and anti-PD-L1 antibody induces better systemic antitumor immunity in dogs with malignant melanoma, the present inventors retrospectively evaluated the response rate of intrathoracic metastatic lesions and overall survival (OS) in dogs with pulmonary metastatic oral malignant melanoma (OMM) (n=39) treated with an anti-PD-L1 antibody (c4G12). In “No RT group” (n=20) consisting of dogs which were considered free from the effect of RT, intrathoracic clinical benefit rate (CBR) was 10.0% (2/20) and median OS was 185 days. In “Previous RT group” (n=9) consisting of dogs which received RT within 8 weeks prior to the first dose of c4G12, CBR was 55.6% (5/9, P=0.016 vs. No RT group) and median OS was 283.5 days (P=0.036 vs. No RT group). In “Concurrent RT group” (n=10) in which c4G12 treatment started within +1 week of the first fraction of RT, CBR was 20.0% (2/10) and median OS was 129 days, with no significant difference compared to No RT group. In the combination therapy, adverse events equivalent to those observed in each monotherapy were observed. Taken together, hypofractionated RT before the initiation of c4G12 treatment can be an effective approach to enhance the therapeutic efficacy of immunotherapy with acceptable safety profiles.

### INTRODUCTION

Melanoma is the most common oral malignancy observed in dogs. The biological behavior of canine oral malignant melanoma (OMM) has been considered locally invasive and highly metastatic^{[1,2]} and is comparable to that of human OMM^{[3]}. Radiation therapy (RT) is an effective treatment for primary OMM and involved lymph nodes. Hypofractionated protocols (e.g., 3-6 once-weekly fractions of 6-9 Gy) have often been used to treat canine OMM^{[4-6]}; however, the optimal dose per fraction, fraction numbers and irradiation intervals have yet to be determined. The reported range of overall response rate of RT on canine malignant melanoma was 73-94%, whereas the progression-free period ranged from 3.6-8.6 months^{[7-11]}. However, the high metastatic potential, especially to the lung, makes it difficult to achieve long-term control of the tumor and ultimately leads to the death of the affected dogs^{[4,12]}. Although the establishment of systemic therapy is necessary to improve the prognosis of malignant melanoma, conventional chemotherapy does not improve the survival time^{[13,14]}. Several immunotherapies have been proposed for the treatment of canine malignant melanoma but, generally, desirable clinical efficacies have not been achieved^{[15]}.

It has been reported that RT enhances anti-tumor immunity not only locally but also systemically. Damage-associated molecular pattern molecules (DAMPs) are released when irradiation causes immunogenic cells death (ICD) in tumor tissues. Cytotoxic T lymphocytes are activated by dendritic cells that have been stimulated by DAMPs via pattern recognition receptors such as toll-like receptors. Subsequently, activated T lymphocytes attack tumor cells systemically^{[16,17]} Enhancement of systemic anti-tumor immunity by irradiation has been recognized as an “abscopal effect” where metastatic lesions outside the irradiation field shrink after local RT. Apart from reports in mouse cancer models, only 46 cases of confirmed abscopal effects have been reported in the medical field from 1969 to 2014^{[18]}. Moreover, the induction of abscopal effect in naturally occurring canine tumors has not been reported^{[19]}. Thus, the enhancement of antitumor immunity by irradiation rarely occurs in spontaneous tumors in both humans and animals. It is considered that the immune evasion mechanisms of tumors can inhibit irradiation-induced antitumor immunity in the vast majority of cancer patients^{[20-24]}.

Programmed cell death 1 (PD-1) and its ligands, PD-ligand 1 (PD-L1) and PD-ligand 2 (PD-L2), are prominent molecules involved in tumor immune evasion and called immune checkpoint molecules. These molecules play important roles in immune suppression to maintain immune homeostasis and to protect the host from excess immune responses or autoimmune diseases. PD-L1 and PD-L2 are type I transmembrane proteins belonging to the B7 family. Although expression of PD-L2 is limited to dendritic cells, macrophages and bone marrow-derived mast cells, PD-L1 is expressed in various cell species including hematopoietic and non-hematopoietic cells^{[25]}. Overexpression of PD-L1 is a common feature in various human tumors, and it causes immune evasion of tumor cells by inhibiting the cell proliferation and cytokine secretion of PD-1-expressing tumor-specific T cells^{[26,27]}. Several types of canine malignant tumors (including squamous cell carcinoma, nasal adenocarcinoma, transitional cell carcinoma, osteosarcoma and malignant melanoma) also express PD-L1, suggesting that canine tumor has an immune evasion mechanism through overexpression of PD-L1^{[28-36]}. The present inventors have reported previously that almost all OMM cases analyzed were expressing PD-L1 (19/20 cases, 95%)^{[35]} and revealed that treatment with a canine chimeric anti-PD-L1 antibody (c4G12) is effective against canine OMM^{[33,35]} Response rate of c4G12 treatment in canine OMM was 14.3% (1/7 dogs)^{[33]}, which was comparable to the response rate of human anti-PD-L1 antibody therapy in patients with advanced malignant melanoma^{[37]}. Although anti-PD-L1 therapy achieved a breakthrough for this extremely progressive tumor, further analysis is needed to improve the response rate and survival benefit.

In irradiation-induced anti-tumor immunity, the PD-1/PD-L1 axis is considered to play a crucial role of suppressing cytotoxic T cell responses within tumor microenvironment, because PD-L1 expression is upregulated after irradiation^{[38]}. Therefore, the combination of RT and anti-PD-1/PD-L1 therapy is expected to induce synergistic antitumor efficacy. Indeed, the combination therapy of RT and immune checkpoint inhibitors (ICIs) induced abscopal effects in mouse cancer models^{[24,39]}, and early clinical studies in humans have demonstrated that non-small-cell lung cancer patients who received previous RT had a significant improvement in overall survival (OS) after anti-PD-1 antibody treatment as compared to those without previous RT^{[40]}.

In this invention, synergistic antitumor efficacy of hypofractionated RT and c4G12 treatment was evaluated in dogs with pulmonary metastatic OMM. To investigate whether combination of RT and c4G12 treatment induce better systemic antitumor immunity, dogs treated with c4G12 at the Hokkaido University Veterinary Teaching Hospital (HUVTH) were retrospectively evaluated for the response rate of intrathoracic (i.e., outside the irradiation field) metastatic lesions and OS. Dogs were divided into three groups based on the presence or absence of RT, as well as the timing of RT, to examine whether the temporal order of the combinational irradiation influences the outcome of c4G12 immunotherapy.

### Materials and Methods

### Entry Requirement

Dogs with pulmonary metastatic OMM (stage IV in the TNM-based staging scheme) that underwent c4G12 treatment at HUVTH between March 2016 and September 2021 were retrospectively evaluated in this study. Pulmonary metastatic sites were detected using chest X-ray or computed tomography (CT) in all dogs. Dogs with concurrent cancer of different origins or severe systemic illness unrelated to the cancer were excluded from the analysis. The clinical study using c4G12 was conducted with the approval of the Institutional Animal Care Committee of Hokkaido University (Approval Nos: 15-0149 and 20-0041) and the Ethics Committee of the Faculty of Veterinary Medicine, Hokkaido University. Prior to enrolment in the clinical study, written informed consent was obtained from the dog owners.

Anti-PD-L1 Antibody (c4G12) Treatment

c4G12 was prepared according to the method disclosed in Japanese Patent No. 6960634. c4G12 treatment was performed as described previously [35]. Dogs received intravenous administration of c4G12 every 2 weeks usually at a dose of 5 mg/kg. In some cases, 2 mg/kg was chosen by veterinarians depending on clinical conditions. For evaluation of adverse events, physical examination, complete blood count, and blood chemistry test were conducted. Adverse events were graded and recorded according to the Veterinary Cooperative Oncology Group-Common Terminology Criteria for Adverse Events (VCOG-CTCAE) v1.1^{[41]}.

### Radiation Therapy (RT)

RT was performed using Elekta Synergy Integrity R LINAC (Elekta A B, Stockholm, Sweden) or TITAN-320S (Shimadzu Industrial System, Kyoto, Japan). The oral tumor and involved lymph nodes, if present, received 4 fractions of RT (approximately 26-32 Gy in total) at one-week intervals. A few dogs (n=3) received RT in other facilities before their first visit to HUVTH, with unspecified protocols and instruments.

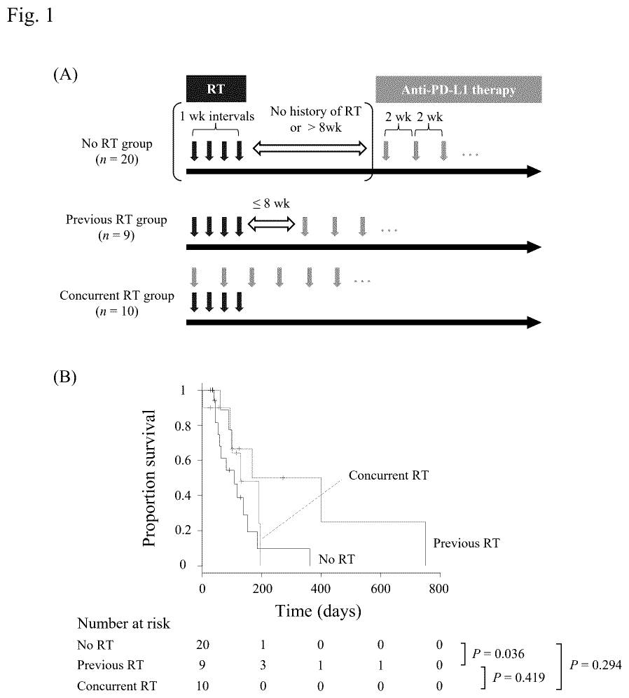

Dogs treated with c4G12 were divided into the following three groups. (1) No RT group: a group consisting of dogs that were considered free from the effect of RT, with no history of RT or the previous RT terminated >8 weeks before the initiation of c4G12 treatment, (2) Previous RT group: consisting of dogs that were treated with RT≤8 weeks prior to the first c4G12 dose, and (3) Concurrent RT group: consisting of dogs that received RT along with the c4G12 treatment, with the first c4G12 dose administered within +1 week of the first fraction of RT (FIG. 1A).

### Evaluation of Intrathoracic Response

Tumor response in the thoracic cavity lesions (i.e., pulmonary metastatic lesions) to c4G12 treatment was recorded according to the response evaluation criteria for solid tumors in dogs (cRECIST) v1.0^{[42]}. Baseline assessments were performed within three weeks of the first c4G12 dose. During treatment, tumors were evaluated at least every 6 weeks using the same modality (chest X-ray or CT). For dogs with non-measurable lesion (i.e., all metastatic lesions in the lung <10 mm on CT or <20 mm on X-ray), tumor response was classified as either complete response (CR, disappearance of all detectable tumors), progressive disease (PD, unequivocal progression of the tumor or appearance of new lesions), or stable disease/partial response (SD/PR, reduction or stable persistence of the tumor: not classified as CR or PD). The clinical benefit rate (CBR) was defined as the percentage of dogs that had the best intrathoracic response of CR, PR, SD, or SD/PR.

### Evaluation of Overall Survival (OS)

OS (days) of dogs was defined as time from the first c4G12 dose to death. In comparison with the historical control group of dogs treated with RT only, OS was defined as time from the first dose of RT to death. Dogs with pulmonary metastatic OMM treated with RT only at HUVTH between 2014 and 2019 were included in the historical control group (n=10, RT-only group). Kaplan-Meier curves were generated to analyze survival time. Dogs that were still alive at the end of the study period were included in the survival analysis as censored data.

### Statistical Analysis

Statistical analyses of binary variables, continuous variables, and survival times were performed using Fisher's exact test, the Kruskal-Wallis test followed by the Steel-Dwass test, and the log-rank test, respectively. The statistical software EZR (version 1.35) was used for all the analyses. p<0.05 was considered statistically significant.

### Results

### Summary of Dogs

In total, 39 dogs with pulmonary metastatic OMM were subjected to analysis, including 26 dogs described in the previous report^{[35]}. There were 12 Miniature dachshunds, 5 mixed-breed dogs, 3 pugs, 3 golden retrievers, 3 toy poodles, 2 American cocker spaniels, 2 Chihuahuas, 2 Labrador retrievers, and one each of the following: beagle, Yorkshire terrier, kaninchen dachshund, papillon, flat-coated retriever, Pomeranian, and Pekingese. Median age was 13 years (range: 8-15 years). There were 21 males (of which 15 castrated) and 18 females (of which 13 spayed). Median body weight was 6.0 kg (range: 2.0-29.0 kg). Primary tumor location included mandible (n=20), maxilla (n=17), and unspecified (n=2) (Table S1). Among 39 dogs, 20 dogs were considered free from the effect of RT (No RT group) at the start of c4G12 treatment. Another 9 dogs were treated with RT≤8 weeks prior to the first c4G12 dose (Previous RT group). The remaining 10 dogs received the first dose of c4G12 within +1 week of the first fraction of RT (Concurrent RT group, see FIG. 1A). No statistically significant differences were observed in sex, age, body weight, and primary tumor location among the treatment groups (Table 1).

### Treatment and Adverse Events

All dogs received at least one dose of c4G12, which was administered every 2 weeks at 2 mg/kg or 5 mg/kg. The median duration of c4G12 treatment was 91 days (range: 2-750 days) with the median number of c4G12 dosage of 6 times (range 1-54 times) (Table S2). In No RT group, adverse events of any grade related to c4G12 treatment occurred in 12 dogs (60%). Although most events were classified as grade 1-2, grade 3 events (pneumonitis or anaphylaxis) occurred in two dogs (10%) in this group. Both grade 3 events were resolved after short-term supportive care. In Previous RT group, adverse events of any grade were observed in four dogs (44%), including grade 3 events (elevation in ALT, AST or lipase) in three dogs (33%). In Concurrent RT group, adverse events of any grade occurred in six dogs (60%), including grade 3 events (elevation in alkaline phosphatase/AST or BUN) in two dogs (20%). None of the grade 3 events in the Previous RT and Concurrent RT groups were associated with clinical symptoms; thus, additional care was not necessary. The frequency of grade 3 adverse events did not differ significantly among the treatment groups (Table 2).

All dogs except for one in Concurrent RT group completed the RT protocol as planned (four fractions of 6.5-8 Gy at one-week intervals). Adverse events related to RT were typically grades 1-2 and were characterized by alopecia, pruritus, skin ulceration and mucositis, which were resolved without treatment. To examine whether the combination therapy increases the risk of side effects associated with RT, the frequency of adverse events related to RT was compared with the corresponding frequency in the historical control group treated with RT alone (n=10, RT Only group, see Table S3). No significant differences were observed in the frequency of adverse events related to RT among RT Only, Previous RT, and Concurrent RT groups (Table S4).

Surgical resection of the metastatic lesion was performed during c4G12 treatment in one dog in No RT group (lymph nodes), two in Previous RT group (lung and eye), and one in Concurrent RT group (spleen). No additional treatment was performed in the remaining 35 dogs (Table S1).

### Intrathoracic Response and Overall Survival (OS)

Intrathoracic metastatic lesions in two dogs responded to c4G12 treatment (one CR and one SD) and CBR was 10.0% (2/20) in No RT group. The dog which achieved CR survived for 362 days, while another dog with SD survived for 152 days. In Previous RT group, four dogs exhibited CR, one dog exhibited SD/PR, and the remaining four dogs exhibited PD. The CBR of intrathoracic metastatic lesions was 55.6% (5/9), which was significantly higher than that of No RT group (P=0.016, see Table 3). Among the dogs with clinical benefit (n=5), the median OS was 399 days (95% confidence interval: 98-NA days). In Concurrent RT group, two dogs exhibited SD/PR and the CBR was 20.0% (2/10), which was not significantly different from that of the other groups (P=0.584 vs. No RT group and P=0.170 vs. Previous RT group). The overall survival periods of the two SD/PR dogs were 131 and 194 days, respectively.

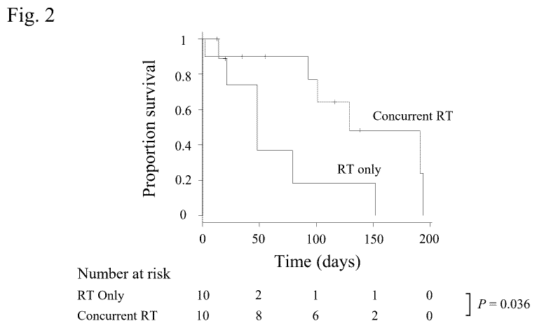

At the end of the study period, one dog in Previous RT group and one in Concurrent RT group were still alive. Twelve dogs were censored because they were lost to follow-up (no record of the date of death). Twenty dogs died of metastasis progression, while four died of local progression of OMM. One dog that experienced CR in Previous RT group died of an unrelated cause (chronic kidney disease) (Table S2). The median OS in all 39 dogs was 129 days (95% confidence interval: 93-185 days). The median OS in No RT group was 185 days (95% confidence interval: 53-152 days). The median OS in Previous OS group was 283.5 days (95% confidence interval: 61-NA days) and was significantly longer than that of No RT group (p=0.036). On the other hand, the median OS in Concurrent RT group was 129 days (95% confidence interval: 2-NA days), with no significant difference in OS as compared to No RT group (p=0.294, see FIG. 1B). However, the OS in Concurrent RT group was significantly longer than that in the historical control group (RT Only group) treated with RT only. The median OS in RT Only group was 48 days (95% confidence interval: 14-NA days, p=0.036, see FIG. 2).

### DISCUSSION

In the present invention, in order to investigate whether anti-PD-L1 therapy combined with RT enhances antitumor effect, the present inventors compared the response rates of pulmonary metastatic lesions located outside of the irradiation field and the OS of dogs with c4G12 treatment among groups divided based on the presence or absence of RT, as well as the timing of RT. In dogs with pulmonary metastatic OMM, intrathoracic tumor response and OS were significantly improved in Previous RT group which received hypofractionated RT before c4G12 treatment, as compared to No RT group. These results strongly suggest that systemic antitumor immunity reinvigorated by anti-PD-L1 therapy was still enhanced via ICD induced by RT. Furthermore, since the concurrent RT initiated with the start of c4G12 treatment did not improve the response rate and OS, it was suggested that the timing of RT is of great importance for achieving synergistic efficacy. As the types, frequency, and severity of adverse events related to c4G12 or RT in combination therapy were similar to those of each treatment alone, hypofractionated RT before c4G12 treatment can be an effective combination therapy with acceptable toxicity to treat canine OMM.

Abscopal effects have been reported in many types of human malignant neoplasms, such as hepatocellular carcinoma, lymphoma, renal cell carcinoma, and melanoma. Although the reported cases were substantially limited, 7 of 46 cases (15.2%) were patients with malignant melanoma. Additionally, the lung is the most common metastatic site, exhibiting an abscopal effect^{[18]}. Thus, dogs with pulmonary metastatic OMM, which the present inventors focused on in this study, are considered to be a suitable model to investigate into abscopal effect and enhancement of antitumor systemic immunity after irradiation. Although abscopal effect itself rarely occurs and has been poorly studied, immunotherapy combined with RT may increase the frequency of cases with an abscopal effect. As ICIs, such as anti-PD-1, anti-PD-L1 or anti-CTLA-4 antibody, enhance systemic antitumor immunity, and local immunosuppression caused by RT is considered to be partly due to the upregulation of immune checkpoint molecules^{[38]}, the combination of ICIs and RT has been gaining attention for its expected synergistic efficacies.

One retrospective study in humans showed the outcomes of 21 patients with advanced melanoma who received RT during treatment with ipilimumab, a CTLA-4 inhibitor. An abscopal effect was observed in 11 patients (52%), nine of whom had PR (43%) and two had SD (10%)^{[44]}. Further, RT during anti-PD-1 therapy achieved a 48% response rate (including CR, PR, and SD) for non-irradiated lesions in advanced melanoma patients^{[45]}. Although the timing of RT and ICI therapy was different, a CBR of 55.6% for non-irradiated lesions of canine OMM in Previous RT group achieved in the present study was equivalent, or more than equivalent, to CBRs achieved in the above combination therapy in human advanced melanoma. Further, the combination of ICIs and RT was associated with prolonged OS as compared to monotherapy in human cancer patients^{[40,46,47]} Consistent with these observations, the OS was significantly longer in Previous RT group than that in No RT group in the present invention. Interestingly, the OS in Concurrent RT group was similar to that of No RT group, with no significant prolongation. However, the OS in Concurrent RT group was significantly longer than that of the historical control group (RT Only group). These results suggest that c4G12 treatment produces survival benefit in any situation, whereas concurrent RT combined with c4G12 treatment does not result in induction of synergistic effects.

The optimal RT protocol (timing, dose, and fractionation) for combined therapy remains a topic of debate^{[48]}. An early study using mouse cancer models revealed that concurrent but not sequential (starting 7 days after the last dose of RT) anti-PD-L1 therapy was associated with improved survival, indicating that concurrent RT induces better synergistic effects^{[49]}. However, in human clinical studies, controversial results have been reported, suggesting that the optimal timing of RT depends on various clinical conditions, including cancer types, patient characteristics, and RT protocols. For example, in patients with metastatic malignant melanoma, concurrent RT combined with anti-PD-1 antibody resulted in a similar OS as compared to sequential therapy (RT followed by anti-PD-1 therapy)^{[50]}. Another study in patients with stage III non-small-cell lung cancer showed that sequential anti-PD-L1 therapy after chemoradiotherapy was associated with better OS than that achieved by chemoradiotherapy alone^{[47,51]}. More recently, a report using mouse models has shown that administration of anti-PD-1 antibody before irradiation increases irradiation-induced death of polyfunctional effector CD8⁺ T cells within the tumor microenvironment, leading to the loss of systemic antitumor immunity^{[52]}. Since the RT protocol used in the present invention was four fractions at one-week intervals and c4G12 was administered every 2 weeks, immune activation by the first two administrations of anti-PD-L1 antibody in Concurrent RT group may have been abrogated by the subsequent fractions of RT, diminishing the synergistic antitumor efficacy. Therefore, the present inventors propose that the optimal timing of RT for canine OMM would be immediately before the initiation of anti-PD-L1 therapy, with the last dose of RT completed before the first dose of anti-PD-L1 antibody.

In conclusion, combination therapy using hypofractionated RT and anti-PD-L1 antibody is a promising strategy to induce robust systemic antitumor immunity in canine malignant melanoma.

TABLE 1
Summary of dogs

| | No RT | Previous RT | Concurrent RT |
|---|---|---|---|
| | (n = 20) | (n = 9) | (n = 10) |
| Breed | | | |
| American cocker spaniel | 1 (5) | 1 (11) | 0 |
| Beagle | 1 (5) | 0 | 0 |
| Chihuahua | 1 (5) | 0 | 1 (10) |
| Flat-coated retriever | 1 (5) | 0 | 0 |
| Golden retriever | 1 (5) | 2 (22) | 0 |
| Kaninchen dachshund | 1 (5) | 0 | 0 |
| Labrador retriever | 1 (5) | 1 (11) | 0 |
| Miniature dachshund | 6 (30) | 2 (22) | 4 (40) |
| Papillon | 1 (5) | 0 | 0 |
| Pekingese | 0 | 1 (11) | 0 |
| Pomeranian | 0 | 1 (11) | 0 |
| Pug | 2 (10) | 0 | 1 (10) |
| Toy poodle | 1 (5) | 0 | 2 (20) |
| Yorkshire terrier | 1 (5) | 0 | 0 |
| Mix | 2 (10) | 1 (11) | 2 (20) |
| Sex | | | |
| Male | 10 (50) | 7 (78) | 4 (40) |
| Castrated | 6 (30) | 5 (56) | 4 (40) |
| Intact | 4 (20) | 2 (22) | 0 |
| Female | 10 (50) | 2 (22) | 6 (60) |
| Spayed | 8 (40) | 1 (11) | 4 (40) |
| Intact | 2 (10) | 1 (11) | 2 (20) |
| Age | | | |
| ≤10 years | 2 (10) | 2 (22) | 1 (10) |
| >10 years | 18 (90) | 7 (78) | 9 (90) |
| Body Weight | | | |
| ≤5 kg | 6 (30) | 2 (22) | 7 (70) |
| 5-10 kg | 10 (50) | 3 (33) | 3 (30) |
| >10 kg | 4 (20) | 4 (44) | 0 |
| Primary tumor site | | | |
| Mandible | 10 (50) | 2 (22) | 5 (50) |
| Maxilla | 8 (40) | 7 (78) | 5 (50) |
| Unspecified | 2 (10) | 0 | 0 |

Number of dogs (%) is shown.

TABLE 2
Table 2. Adverse events related to c4G12 treatment.

| | No RT | | Previous RT | | Concurrent RT | |
|---|---|---|---|---|---|---|
| | (n = 20) | | (n = 9) | | (n = 10) | |
| | Any grade | Grade 3 | Any grade | Grade 3 | Any grade | Grade 3 |
| Any | 12 (60) | 2 (10) | 4 (44) | 3 (33) | 6 (60) | 2 (20) |
| Anorexia | 0 | 0 | 1 (11) | 0 | 0 | 0 |
| Diarrhoea | 2 (10) | 0 | 1 (11) | 0 | 2 (20) | 0 |
| Vomiting | 3 (15) | 0 | 2 (22) | 0 | 2 (20) | 0 |
| Alkaline | 1 (5) | 0 | 0 | 0 | 2 (20) | 1 (10) |
| phosphatase | | | | | | |
| ALT | 5 (25) | 0 | 3 (33) | 1 (11) | 1 (10) | 0 |
| AST | 0 | 0 | 3 (33) | 1 (11) | 1 (10) | 1 (10) |
| BUN | 0 | 0 | 0 | 0 | 2 (20) | 1 (10) |
| Albumin, low | 0 | 0 | 1 (11) | 0 | 0 | 0 |
| Glucose, low | 0 | 0 | 1 (11) | 0 | 0 | 0 |
| Lipase | 2 (10) | 0 | 1 (11) | 1 (11) | 1 (10) | 0 |
| CPK | 0 | 0 | 1 (11) | 0 | 0 | 0 |
| Conjunctivitis | 1 (5) | 0 | 0 | 0 | 0 | 0 |
| Thrombocytopenia | 2 (10) | 0 | 0 | 0 | 0 | 0 |
| Pancreatitis | 0 | 0 | 0 | 0 | 1 (10) | 0 |
| Pneumonia | 1 (5) | 1 (5) | 0 | 0 | 0 | 0 |
| Allergic reaction | 0 | 0 | 0 | 0 | 1 (10) | 0 |
| Anaphylaxis | 1 (5) | 1 (5) | 0 | 0 | 0 | 0 |

Number of dogs with event (%) is shown.
Adverse events that were considered to be related to c4G12 treatment by the inventors were
listed.
Grading was performed in accordance with VCOG-CTCAE v1.1 [41].
ALT, alanine aminotransferase; AST, aspartate aminotransferase; BUN, blood urea nitrogen;
CPK, creatine phosphokinase.

TABLE 3
Intrathoracic response to c4G12 treatment.

| | No RT | Previous RT | Concurrent RT |
|---|---|---|---|
| | (n = 20) | (n = 9) | (n = 10) |
| CR | 1 (5) | 4 (44) | 0 |
| SD or SD/PR^{a} | 1 (5) | 1 (11) | 2 (20) |
| PD | 15 (75) | 4 (44) | 6 (60) |
| NE | 3 (15) | 0 | 2 (20) |
| Clinical benefit^{b} | 2 (10) | 5 (56)^{c} | 2 (20) |

Number of dogs (%) is shown.
Tumor response was recorded in accordance with cRECIST v1.0 [42].
^{a}Non-CR/non-PD in dogs with non-measurable pulmonary lesions alone at the time of baseline evaluation.
^{b}Clinical benefit = CR + PR + SD (SD/PR)
^{c}P < 0.05 by Fisher's exact test (vs. No RT group).
CR, complete response; PR, partial response; SD, stable disease; PD, progressive disease; NE, not evaluable.

TABLE S1
Table S1. Summary of c4G12-treated dogs.

| | | | | Body | Site of | | |
|---|---|---|---|---|---|---|---|
| Dog | | | Age | weight | primary | Prior | Concurrent |
| # | Breed | Sex | (Year) | (kg) | tumor | therapy | therapy |
| 1 | Pug | Male, | 11 | 8.4 | Mandible | Surgery | None |
| | | castrated | | | | | |
| 2 | Miniature | Male, | 14 | 8.8 | Maxilla | Radiation | None |
| | dachshund | castrated | | | | | |
| 3 | Golden | Male | 10 | 26.4 | Maxilla | Surgery | None |
| | retriever | castrated | | | | | |
| 4 | Beagle | Female, | 11 | 9.7 | Maxilla | Surgery | Surgery |
| | | spayed | | | | | |
| 5 | American cocker | Male, | 13 | 8.9 | Mandible | Surgery | None |
| | spaniel | castrated | | | | | |
| 6 | Yorkshire terrier | Male | 14 | 2.0 | Mandible | Surgery | None |
| 7 | Kaninchen | Female, | 13 | 5.7 | Mandible | Surgery, | None |
| | dachshund | spayed | | | | chemotherapy | |
| 8 | Miniature | Male | 13 | 6.5 | Mandible | Surgery | None |
| | dachshund | | | | | | |
| 9 | Mix | Female | 12 | 16.8 | Maxilla | Surgery | None |
| 10 | Miniature | Male | 13 | 4.7 | Unspecified | Surgery, | None |
| | dachshund | | | | | radiation | |
| 11 | Miniature | Female | 15 | 4.4 | Mandible | Surgery | None |
| | dachshund | | | | | | |
| 12 | Toy poodle | Female, | 13 | 4.6 | Maxilla | Radiation | None |
| | | spayed | | | | | |
| 13 | Miniature | Male | 14 | 5.2 | Maxilla | Radiation | None |
| | dachshund | | | | | | |
| 14 | Chihuahua | Female, | 13 | 2.3 | Mandible | Surgery | None |
| | | spayed | | | | | |
| 15 | Pug | Female, | 14 | 7.6 | Mandible | Surgery | None |
| | | spayed | | | | | |
| 16 | Papillon | Male, | 14 | 3.7 | Unspecified | Surgery | None |
| | | castrated | | | | | |
| 17 | Flat-coated | Female, | 8 | 26.7 | Maxilla | Surgery | None |
| | retriever | spayed | | | | | |
| 18 | Miniature | Male, | 14 | 8.3 | Mandible | None | None |
| | dachshund | castrated | | | | | |
| 19 | Mix | Female, | 15 | 9.0 | Maxilla | Surgery | None |
| | | spayed | | | | | |
| 20 | Labrador | Female, | 12 | 29.0 | Mandible | Surgery | None |
| | retriever | spayed | | | | | |
| 21 | Miniature | Male, | 14 | 5.7 | Maxilla | Radiation | None |
| | dachshund | castrated | | | | | |
| 22 | Pomeranian | Male | 12 | 2.6 | Maxilla | Surgery, | None |
| | | | | | | radiation | |
| 23 | Pekingese | Male, | 8 | 6.5 | Maxilla | Radiation | None |
| | | castrated | | | | | |
| 24 | Mix | Male, | 11 | 15.4 | Maxilla | Radiation | None |
| | | castrated | | | | | |
| 25 | Labrador | Female | 11 | 22.5 | Mandible | Radiation | None |
| | retriever | | | | | | |
| 26 | American cocker | Male | 10 | 8.0 | Maxilla | Radiation | None |
| | spaniel | | | | | | |
| 27 | Miniature | Female, | 15 | 4.2 | Mandible | Surgery, | Surgery |
| | dachshund | spayed | | | | radiation | |
| 28 | Golden | Male | 14 | 29.0 | Maxilla | Surgery, | Surgery |
| | retriever | castrated | | | | radiation | |
| 29 | Golden | Male, | 11 | 26.0 | Maxilla | Surgery, | None |
| | retriever | castrated | | | | radiation | |
| 30 | Miniature | Female, | 10 | 4.8 | Maxilla | None | Radiation |
| | dachshund | spayed | | | | | |
| 31 | Miniature | Male, | 15 | 4.7 | Mandible | Surgery | Radiation |
| | dachshund | castrated | | | | | |
| 32 | Pug | Female | 14 | 6.0 | Mandible | None | Radiation |
| 33 | Miniature | Female, | 15 | 4.2 | Mandible | None | Radiation |
| | dachshund | spayed | | | | | |
| 34 | Miniature | Male, | 14 | 4.6 | Mandible | None | Radiation |
| | dachshund | castrated | | | | | |
| 35 | Toy poodle | Female | 13 | 3.7 | Maxilla | None | Radiation |
| 36 | Mix | Female, | 14 | 6.6 | Maxilla | None | Radiation |
| | | spayed | | | | | |
| 37 | Chihuahua | Male, | 13 | 4.7 | Maxilla | None | Radiation |
| | | castrated | | | | | |
| 38 | Toy poodle | Female, | 14 | 5.5 | Maxilla | None | Radiation, |
| | | spayed | | | | | surgery |
| 39 | Mix | Male, | 13 | 3.6 | Mandible | None | Radiation |
| | | castrated | | | | | |

TABLE S2
Table S2. Summary of c4G12 treatment.

| | c4G12 | No. of | | Timing | Overall | | Cause |
|---|---|---|---|---|---|---|---|
| Dog | treatment | c4G12 | | of | survival | Intrathoracic | of |
| # | duration | doses | Dosage | RT | (days) | responsec | death |
| 1 | 91 | 7 | Weeks 0-8: 2 | No RT | 91ª | PD | NA (Censored) |
| | | | mg/kg, then 5 | | | | |
| | | | mg/kg | | | | |
| 2 | 42 | 3 | 2 mg/kg | No RT | 81 | PD | Local progression |
| 3 | 36 | 3 | 2 mg/kg | No RT | 36ª | PD | NA (Censored) |
| 4 | 335 | 25 | Weeks 0-12: 5 | No RT | 362 | CR | Advanced |
| | | | mg/kg, then 2 | | | | metastasis |
| | | | mg/kg | | | | |
| 5 | 98 | 7 | 5 mg/kg | No RT | 108 | PD | Local progression |
| 6 | 44 | 4 | 5 mg/kg | No RT | 44 | PD | Advanced |
| | | | | | | | metastasis |
| 7 | 35 | 2 | 5 mg/kg | No RT | 35ª | NE | NA (Censored) |
| 8 | 42 | 3 | 5 mg/kg | No RT | 44ª | PD | NA (Censored) |
| 9 | 128 | 9 | 5 mg/kg | No RT | 128ª | PD | NA (Censored) |
| 10 | 28 | 3 | 5 mg/kg | No RT | 28ª | NE | NA (Censored) |
| 11 | 28 | 2 | 5 mg/kg | No RT | 44 | PD | Advanced |
| | | | | | | | metastasis |
| 12 | 35 | 3 | 5 mg/kg | No RT | 58 | PD | Advanced |
| | | | | | | | metastasis |
| 13 | 118 | 8 | 5 mg/kg | No RT | 118 | PD | Advanced |
| | | | | | | | metastasis |
| 14 | 185 | 13 | 5 mg/kg | No RT | 185 | PD | Advanced |
| | | | | | | | metastasis |
| 15 | 26 | 2 | 5 mg/kg | No RT | 40 | PD | Advanced |
| | | | | | | | metastasis |
| 16 | 15 | 1 | 5 mg/kg | No RT | 42ª | PD | NA (Censored) |
| 17 | 138 | 10 | 2 mg/kg | No RT | 138 | PD | Advanced |
| | | | | | | | metastasis |
| 18 | 42 | 3 | 5 mg/kg | No RT | 53 | PD | Advanced |
| | | | | | | | metastasis |
| 19 | 152 | 11 | 5 mg/kg | No RT | 152 | SD | Advanced |
| | | | | | | | metastasis |
| 20 | 14 | 2 | 2 mg/kg | No RT | 63 | NE | Advanced |
| | | | | | | | metastasis |
| 21 | 82 | 6 | 5 mg/kg | Previous RT | 88 | PD | Advanced |
| | | | | | | | metastasis |
| 22 | 168 | 11 | 5 mg/kg | Previous RT | 168 | CR | Chronic kidney |
| | | | | | | | disease |
| 23 | 271 | 19 | 5 mg/kg | Previous RT | 271ª | CR | NA (Censored) |
| 24 | 61 | 5 | 5 mg/kg | Previous RT | 61 | PD | Advanced |
| | | | | | | | metastasis |
| 25 | 98 | 6 | 2 mg/kg | Previous RT | 98 | SD/PR | Local progression |
| 26 | 101 | 7 | 5 mg/kg | Previous RT | 101ª | PD | NA (Censored) |
| 27 | 750 | 54 | 5 mg/kg | Previous RT | 750 | CR | Advanced |
| | | | | | | | metastasis |
| 28 | 399 | 28 | Weeks 0-42: 2 | Previous RT | 399 | CR | Advanced |
| | | | mg/kg, then 5 | | | | metastasis |
| | | | mg/kg | | | | |
| 29 | 124 | 9 | 2 mg/kg | Previous RT | 124b | PD | Alive (Censored) |
| 30 | 2 | 1 | 5 mg/kg | Concurrent RT | 2 | NE | Advanced |
| | | | | | | | metastasis |
| 31 | 89 | 6 | 5 mg/kg | Concurrent RT | 93 | PD | Advanced |
| | | | | | | | metastasis |
| 32 | 188 | 13 | 5 mg/kg | Concurrent RT | 194 | SD/PR | Local progression |
| 33 | 28 | 3 | 5 mg/kg | Concurrent RT | 28ª | PD | NA (Censored) |
| 34 | 56 | 4 | 5 mg/kg | Concurrent RT | 56ª | PD | NA (Censored) |
| 35 | 129 | 10 | 5 mg/kg | Concurrent RT | 129 | PD | Advanced |
| | | | | | | | metastasis |
| 36 | 116 | 9 | 5 mg/kg | Concurrent RT | 116ª | PD | NA (Censored) |
| 37 | 175 | 11 | 5 mg/kg | Concurrent RT | 191 | PD | Advanced |
| | | | | | | | metastasis |
| 38 | 131 | 8 | 5 mg/kg | Concurrent RT | 131b | SD/PR | Alive (Censored) |
| 39 | 20 | 2 | 5 mg/kg | Concurrent RT | 101 | NE | Advanced |
| | | | | | | | metastasis |

Overall survival (days) was defined as time from the first c4G12 dose to death.
Censored due to ªloss to follow-up or balive at the end of study period.
`Tumor response was recorded according to cRECIST v1.0 [42].
CR, complete response; PR, partial response; SD, stable disease; PD, progressive disease;
NA, not applicable.

TABLE S3
Summary of dogs in RT Only group

| | | | Overall survival |
|---|---|---|---|
| | | | from the |
| | | Age | beginning of RT |
| Breed | Sex | (Year) | (days) |
| Golden retriever | Female, spayed | 14 | 13* |
| Miniature dachshund | Male, castrated | 14 | 48 |
| Miniature dachshund | Female, spayed | 13 | 14 |
| Miniature dachshund | Female, spayed | 14 | 48 |
| Mix | Male, castrated | 13 | 21 |
| Chihuahua | Male, castrated | 13 | 152 |
| Miniature dachshund | Male | 13 | 79 |
| Miniature dachshund | Male | 16 | 20* |
| Pug | Female, spayed | 14 | 14* |
| Miniature schnauzer | Male | 12 | 21* |

Overall survival (days) was defined as time from the first dose of radiation therapy to death.
*Censored due to loss to follow-up.

TABLE S4
Table S4. Adverse events related to RT

| | RT only | | Previous RT | | Concurrent RT | |
|---|---|---|---|---|---|---|
| | (n =10) | | (n = 9) | | (n = 10) | |
| | Any | Grade | Any | Grade | Any | Grade |
| | grade | 3 | grade | 3 | grade | 3 |
| Any | 2 (20) | 0 | 3 (33) | 0 | 6 (60) | 1 (10) |
| Alopecia | 1 (10) | 0 | 1 (11) | 0 | 2 (20) | 0 |
| Pruritus | 1 (10) | 0 | 1 (11) | 0 | 2 (20) | 0 |
| Mucositis/ | 0 | 0 | 1 (11) | 0 | 1 (10) | 0 |
| stomatitis | | | | | | |
| Skin | 0 | 0 | 2 (22) | 0 | 0 | 0 |
| ulceration | | | | | | |
| AST | NE | NE | NE | NE | 2 (20) | 1 (10) |

Number of dogs with event (%) is shown.
Adverse events that were considered to be related to RT by the inventors were listed.
Grading was performed in accordance with VCOG-CTCAE v1.1 [41].
AST, aspartate aminotransferase; NE, not evaluated.

### REFERENCES

1. Bostock, D. E. Prognosis after surgical excision of canine melanomas. Vet. Pathol. 16, 32-40 (1979).
2. Millanta, F. et al. Proliferation activity in oral and cutaneous canine melanocytic tumours: correlation with histological parameters, location, and clinical behaviour. Res. Vet. Sci. 73, 45-51 (2002).
3. Vail, D. M. & MacEwen, E. G. Spontaneously occurring tumors of companion animals as models for human cancer. Cancer Invest. 18, 781-792 (2000).
4. Freeman, K. P., Hahn K. A., Harris F. D. & King G. K. Treatment of dogs with oral melanoma by hypofractionated radiation therapy and platinum-based chemotherapy (1987-1997). J. Vet. Intern. Med. 17:96-101 (2003).
5. Murphy, S. et al. Oral malignant melanoma—the effect of coarse fractionation radiotherapy alone or with adjuvant carboplatin therapy. Vet. Comp. Oncol. 3, 222-229 (2005).
6. Dank, G. et al. Use of adjuvant carboplatin for treatment of dogs with oral malignant melanoma following surgical excision. Vet. Comp. Oncol. 12, 78-84 (2014).
7. Cancedda, S. et al. Efficacy and side effects of radiation therapy in comparison with radiation therapy and temozolomide in the treatment of measurable canine malignant melanoma. Vet. Com. Oncol. 14, e146-e157 (2016).
8. Bateman, K. E. Catton, P. A., Pennock, P. W. & Kruth, S. A. 0-7-21 radiation therapy for the treatment of canine oral melanoma. J. Vet. Intern. Med. 8, 267-272 (1994).
9. Blackwood, L. & Dobson, J. M. Radiotherapy of oral malignant melanomas in dogs. J. Am. Vet. Med. Assoc. 209, 98-102 (1996).
10. Kawabe, M. et al. Outcomes of dogs undergoing radiotherapy for treatment of oral malignant melanoma: 111 cases (2006-2012). J. Am. Vet. Med. Assoc. 247, 1146-1153 (2015).
11. Tollett, M. A., Duda, L., Brown, D. C. & Krick E. L. Palliative radiation therapy for solid tumors in dogs: 103 cases (2007-2011). J. Am. Vet. Med. Assoc. 248, 72-82 (2016).
12. Proulx, D. R. et al. A retrospective analysis of 140 dogs with oral melanoma treated with external beam radiation. Vet. Radiol. Ultrasound 44, 352-359 (2003).
13. Rassnick, K. M. et al. Use of carboplatin for treatment of dogs with malignant melanoma: 27 cases (1989-2000). J. Am. Vet. Med. Assoc. 218, 1444-1448 (2001).
14. Boria, P. A. et al. Evaluation of cisplatin combined with piroxicam for the treatment of oral malignant melanoma and oral squamous cell carcinoma in dogs. J. Am. Vet. Med. Assoc. 224, 388-394 (2004).
15. Almela, R. M. & Anson, A. A review of immunotherapeutic strategies in canine malignant melanoma. Vet. Sci. 6, 15 (2019).
16. Palata, O. et al. Radiotherapy in combination with cytokine treatment. Front. Oncol. 9, 367 (2019).
17. Frey, B. et al. Induction of abscopal anti-tumor immunity and immunogenic tumor cell death by ionizing irradiation-implications for cancer therapies. Curr. Med. Chem. 19, 1751-1764 (2912).
18. Abuodeh, Y. Venkat, P. & Kim, S. Systematic review of case reports on the abscopal effect. Curr. Probl. Cancer. 40, 25-37 (2016).
19. Fan T. M. & Selting, K. A. Exploring the potential utility of pet dogs with cancer for studying radiation-induced immunogenic cell death strategies. Front. Oncol. 8, 680 (2019).
20. Ahn, G-O. et al. Inhibition of Mac-1 (CD11b/CD18) enhances tumor response to radiation by reducing myeloid cell recruitment. Proc. Natl. Acad. Sci. U.S.A. 107, 8363-8368 (2010).
21. Chiang, C-S. et al. Irradiation promotes an M2 macrophage phenotype in tumor hypoxia. Front. Oncol. 2, 89 (2012).
22. Crittenden, M. R. et al. Expression of NF-kappaB p50 in tumor stroma limits the control of tumors by radiation therapy. PLOS ONE 7, e39295 (2012).
23. Vanpouille-Box, C. et al. TGFß Is a Master Regulator of Radiation Therapy-Induced Antitumor Immunity. Cancer Res. 75, 2232-2242 (2015).
24. Park, S. S. et al. PD-1 restrains radiotherapy-induced abscopal effect. Cancer Immunol. Res. 3, 610-619 (2015).
25. Keir, M. E., Butte M. J., Freeman, G. J. & Sharpe A. H. PD-1 and its ligands in tolerance and immunity. Annu. Rev. Immunol. 26, 677-704 (2008).
26. Ganss, R. & Hanahan, D. Tumor microenvironment can restrict the effectiveness of activated antitumor lymphocytes. Cancer Res. 58, 4673-4681 (1998).
27. Sznol, M. & Chen, L. Antagonist antibodies to PD-1 and B7-H1 (PD-L1) in the treatment of advanced human cancer. Clin Cancer Res. 19, 1021-1034 (2013).
28. Maekawa, N. et al. Expression of PD-L1 on canine tumor cells and enhancement of IFN-γ production from tumor-infiltrating cells by PD-L1 blockade. PLOS One 9, e98415 (2014).
29. Hartley, G. et al. Immune regulation of canine tumour and macrophage PD-L1 expression. Vet. Comp. Oncol. 15, 534-549 (2017).
30. Shosu, K. et al. Programmed cell death ligand 1 expression in canine cancer. In Vivo 30, 195-204 (2016).
31. Maekawa, N. et al. Immunohistochemical analysis of PD-L1 expression in canine malignant cancers and PD-1 expression on lymphocytes in canine oral melanoma. PLOS One 11, e0157176 (2016).
32. Kumar, S. R. et al. Programmed death ligand 1 is expressed in canine B cell lymphoma and downregulated by MEK inhibitors. Vet. Comp. Oncol. 15, 1527-1536 (2017).
33. Maekawa, N. et al. A canine chimeric monoclonal antibody targeting PD-L1 and its clinical efficacy in canine oral malignant melanoma or undifferentiated sarcoma. Sci. Rep. 7, 8951 (2017).
34. Hartley, G., Elmslie, R., Dow, S. & Guth, A. Checkpoint molecule expression by B and T cell lymphomas in dogs. Vet. Comp. Oncol. 16, 352-360 (2018).
35. Maekawa, N. et al. PD-L1 immunohistochemistry for canine cancers and clinical benefit of anti-PD-L1 antibody in dogs with pulmonary metastatic oral malignant melanoma. NPJ Precis. Oncol. 5, 10 (2021).
36. Gulay, K. C. M. et al. Hemangiosarcoma cells induce M2 polarization and PD-L1 expression in macrophages. Sci. Rep. 12:2124 (2022).
37. Brahmer, J. R. et al. Safety and activity of anti-PD-L1 antibody in patients with advanced cancer. N. Engl. J. Med., 366, 2455-2465 (2012).
38. Sato, H., Okonogi, N. &. Nakano T. Rationale of combination of anti-PD-1/PD-L1 antibody therapy and radiotherapy for cancer treatment. Int. J. Clin. Oncol. 25, 801-809 (2020).
39. Takahashi, Y. et al. Radiation enhanced the local and distant anti-tumor efficacy in dual immune checkpoint blockade therapy in osteosarcoma. PLOS ONE 12, e0189697 (2017).
40. Shaverdian, N. et al. Previous radiotherapy and the clinical activity and toxicity of pembrolizumab in the treatment of non-small-cell lung cancer: a secondary analysis of the KEYNOTE-001 phase 1 trial. Lancet Oncol. 18, 895-903 (2017).
41. Veterinary Cooperative Oncology Group (VCOG). Veterinary cooperative oncology group-common terminology criteria for adverse events (VCOG-CTCAE) following chemotherapy or biological antineoplastic therapy in dogs and cats v1.1. Vet. Comp. Oncol. 14, 417-446 (2016).
42. Nguyen, S. M., Thamm, D. H., Vail, D. M. & London, C. A. Response evaluation criteria for solid tumours in dogs (v1.0): A Veterinary Cooperative Oncology Group (VCOG) consensus document. Vet. Comp. Oncol. 13, 176-183 (2015).
43. Kanda, Y. Investigation of the freely available easy-to-use software ‘EZR’ for medical statistics. Bone Marrow Transplant 48, 452-458 (2013).
44. Grimaldi, A. M. et al. Abscopal effects of radiotherapy on advanced melanoma patients who progressed after ipilimumab immunotherapy. Oncoimmunology 3, e28780 (2014).
45. Roger, A. et al. Efficacy of combined hypo-fractionated radiotherapy and anti-PD-1 monotherapy in difficult-to-treat advanced melanoma patients. Oncoimmunology 7, e1442166 (2018).
46. Yamaguchi, O. et al. Radiotherapy is an independent prognostic marker of favorable prognosis in non-small cell lung cancer patients after treatment with the immune checkpoint inhibitor, nivolumab. Thorac. Cancer 10, 992-1000 (2019).
47. Antonia, S. J. et al. Overall survival with durvalumab after chemoradiotherapy in stage III NSCLC. N. Engl. J. Med. 379, 2342-2350 (2018).
48. Kong, Y. et al. Optimizing the treatment schedule of radiotherapy combined with anti-PD-1/PD-L1 immunotherapy in metastatic cancers. Front. Oncol. 11, 638873 (2021).
49. Dovedi, S. J. et al. Acquired resistance to fractionated radiotherapy can be overcome by concurrent PD-L1 blockade. Cancer Res. 74, 5458-5468 (2014).
50. Liniker, E. et al. Activity and safety of radiotherapy with anti-PD-1 drug therapy in patients with metastatic melanoma. Oncoimmunology 5, e1214788 (2016).
51. Gray, J. E. et al. Three-year overall survival with durvalumab after chemoradiotherapy in Stage III NSCLC-update from PACIFIC. J. Thorac. Oncol. 15, 288-293 (2020).
52. Wei, J. et al. Sequence of aPD-1 relative to local tumor irradiation determines the induction of abscopal antitumor immune responses. Sci. Immunol. 6, eabg0117 (2021).

All publications, patents and patent applications cited herein are incorporated herein by reference in their entirety.

### INDUSTRIAL APPLICABILITY

The present invention is applicable to treatment of cancer in non-human mammals.

### Sequence Listing Free Text

<SEQ ID NO: 1> shows the amino acid sequence
of the light chain of c4G12.
MESQTHVLISLLLSVSGTYGDIAITQSPSSVAVSVGETVTLSCKSSQSLL
YSENQKDYLGWYQQKPGQTPKPLIYWATNRHTGVPDRFTGSGSGTDFTLI
ISSVQAEDLADYYCGQYLVYPFTFGPGTKLELKQPKASPSVTLFPPSSEE
LGANKATLVCLISDFYPSGVTVAWKASGSPVTQGVETTKPSKQSNNKYAA
SSYLSLTPDKWKSHSSFSCLVTHEGSTVEKKVAPAECS
<SEQ ID NO: 2> shows the amino acid sequence
of the heavy chain of c4G12.
MGWSQIILFLVAAATCVHSQVQLQQSGAELVKPGSSVKISCKASGYTFTS
NFMHWVKQQPGNGLEWIGWIYPEYGNTKYNQKFDGKATLTADKSSSTAYM
QLSSLTSEDSAVYFCASEEAVISLVYWGQGTLVTVSSASTTAPSVFPLAP
SCGSTSGSTVALACLVSGYFPEPVTVSWNSGSLTSGVHTFPSVLQSSGLY
SLSSTVTVPSSRWPSETFTCNVVHPASNTKVDKPVPKESTCKCISPCPVP
ESLGGPSVFIFPPKPKDILRITRTPEITCVVLDLGREDPEVQISWFVDGK
EVHTAKTQPREQQFNSTYRVVSVLPIEHQDWLTGKEFKCRVNHIGLPSPI
ERTISKARGQAHQPSVYVLPPSPKELSSSDTVTLTCLIKDFFPPEIDVEW
QSNGQPEPESKYHTTAPQLDEDGSYFLYSKLSVDKSRWQQGDTFTCAVMH
EALQNHYTDLSLSHSPGK

1. A method of treating cancer in non-human mammals, comprising initiating treatment with a PD-1/PD-L1 inhibitor after conducting radiation therapy.

2. A pharmaceutical composition for treating cancer in non-human mammals, wherein the composition comprises a PD-1/PD-L1 inhibitor and administration of the composition is initiated after conducting radiation therapy.

3. A PD-1/PD-L1 inhibitor for use in a method of treating cancer in non-human mammals, comprising initiating treatment with the PD-1/PD-L1 inhibitor after conducting radiation therapy.
